(19) United States
(12) Patent Application Publication
(10) Pub. No.: US 2022/0022743 A1
(43) Pub. Date: Jan. 27, 2022

(54) PORTABLE DEVICE FOR VISUAL FUNCTION TESTING

(71) Applicant: Ocular Mobile Innovations Inc., Ottawa (CA)

(72) Inventors: Rejean Munger, Ottawa (CA); Martin Edson Rivers, Jr., Ottawa (CA)

(73) Assignee: Ocular Mobile Innovations Inc., Ottawa, ON (CA)

(21) Appl. No.: 17/298,799

(22) PCT Filed: Dec. 9, 2019
(86) PCT No.: PCT/CA2019/051763

§ 371 Date: Jun. 1, 2021

Related U.S. Application Data
(60) Provisional application No. 62/776,609, filed on Dec. 7, 2018.

(51) Int. Cl. A61B 3/14; A61B 3/00; A61B 3/135; A61B 3/103; A61B 3/11

(52) U.S. Cl. CPC A61B 3/14; A61B 3/0008; A61B 3/112; A61B 3/103; A61B 3/135

(57) ABSTRACT

The present invention relates to a device and method for vision screening, impairment and concussion detection. The device for visual function testing comprises optic components, projection optics and an image capture component.

### FIELD OF THE INVENTION

The present invention relates generally to the field of ocular examination. More particularly, the present invention relates to a device and method for vision screening, impairment and concussion detection.

### BACKGROUND

An examination of the eyes provides an assessment of vision and can detect potentially treatable blinding eye diseases. In addition, a number of systemic diseases have ocular manifestations. As such, an eye examination may be useful in the detection of certain systemic diseases. Furthermore, drugs and alcohol can affect the eye, for example, affect pupil diameter and eye movement. Likewise, a concussion may also exhibit ocular manifestations. Furthermore, an ocular examination can detect immediate affectations that could lead to less than optimal task performance, for example lack of sleep or oxygen deprivation. Accordingly, an examination may be used to screen for impairment and concussions.

An eye examination may include a variety of tests depending on the purpose of the examination which may include screening, diagnostic, treatment monitoring. A minimal eye examination typically consists of tests for visual acuity, pupil function, and extraocular muscle motility, as well as direct ophthalmoscopy through either an undilated or dilated pupil. Examinations are usually performed in a professional medical office which is a barrier to access. Typical examinations, including screening examinations, require the use of multiple single purpose instruments that are not portable. Providing a single portable device that can perform multiple eye tests at patient's location would dramatically increase access to eye examinations in general and more importantly to screening eye examinations.

Mobile technology can provide increased access to eye examinations. Mobile devices for screening the eyes are known in the art. See, for example, U.S. Pat. Nos. 9,237,846; 9,402,538; 9,408,535; US20170027440; U.S. Pat. Nos. 9,655,517 and 9,357,966.

### BRIEF DESCRIPTION OF THE FIGURES

FIG. 1 provides an illustration of the platform for visual function testing of various embodiments of the present invention. OD and OS refer to the right and left eye in that order. LCD's are liquid crystal displays, MEMS are micro-Electro-Mechanical-Systems, BS is a beam splitter.

FIG. 2 provides details of the testing capabilities of various components of the platform for visual function testing of an embodiment of the present invention.

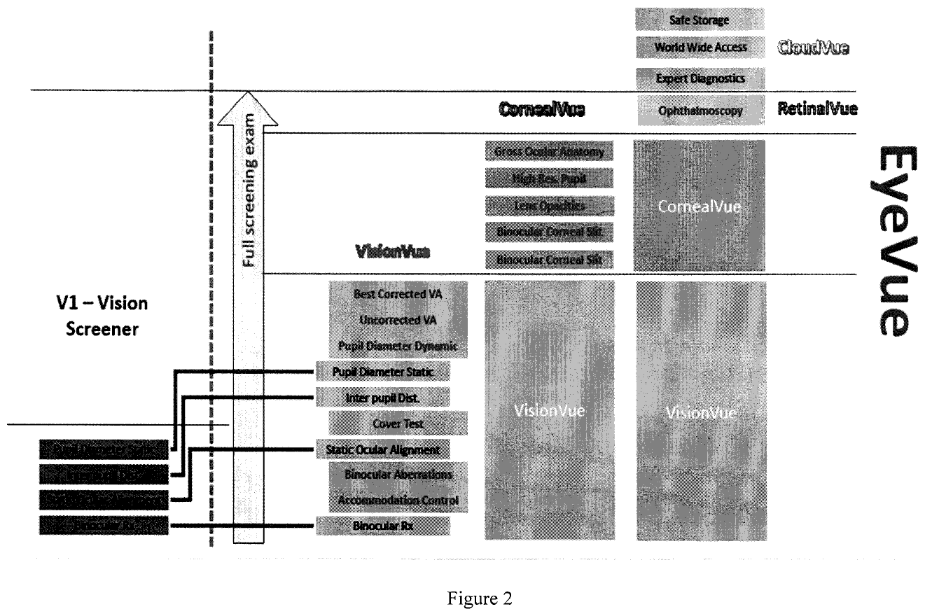

FIG. 3 provides details with respect to the slit lamp projector of an embodiment of the present invention.

FIG. 4 provides a Venn diagram detailing the relations between vision screening, concussion screening and impairment screening.

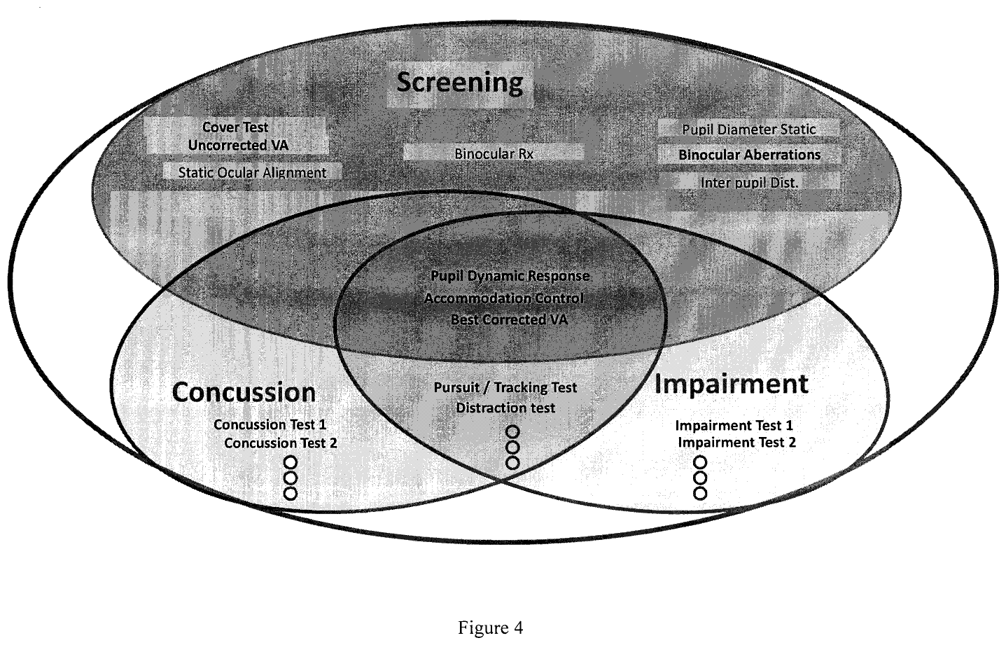

FIG. 5 provides details with respect to the vision screening of an embodiment of the present invention.

FIG. 6 provides details with respect to the functional tests of an embodiment of the present invention. In particular, FIG. 6 illustrates the use of the device as an impairment tester.

### SUMMARY OF THE INVENTION

An object of the present invention is to provide a portable device for visual function testing. In accordance with an aspect of the present invention, there is provided a device for visual function testing comprising optic components, projection optics and an image capture component.

In certain embodiments, the optical components may comprise one or more means to generate an image and/or to provide illumination.

In certain embodiments, the one or means are selected from the group consisting of LED displays, Organic LED (OLED) displays, Liquid Crystal Displays (LCD), micro-electro-mechanical-systems (MEMS) based microprojectors, quantum dot displays and combinations thereof may be used to generate the image and/or provide illumination.

In certain embodiments, separate means are used to generate the image and/or provide illumination are used for each eye.

In certain embodiments, a single means is used in combination with additional components for display for both eyes, and wherein said additional components comprise prism(s), mirror(s) and/or beam splitter(s).

In certain embodiments, the image capture component is configured to capture images of both eyes simultaneously.

In certain embodiments, the image capture component comprises one or more digital cameras.

In certain embodiments, the device further comprises projection optics which provide magnification/minification of the generated image for the eye; focuses the image at a selected distance; and/or limit the field of view (area of projection) so it is limited to the targeted eye.

In certain embodiments, the projection optics comprise a focussing element.

### DETAILED DESCRIPTION

The present invention provides a device and method of ocular testing. The functional vision testing platform of the present invention may be used to perform a broad range of vision tests, which can be selected as single tests or in groups of test for vision screening, impairment detection and concussion detection. The device of the present invention projects independent images to each eye, optionally simultaneously. This projection may be used to illuminate the eye and/or stimulate vision based on psychophysical science.

Accordingly, the device of the present invention comprises optical components, projection optics and an image capture component.

The optical components comprise one or more means to generate an image and/or to provide illumination. A variety of different technologies including but not limited to LED displays, Organic LED (OLED) displays, Liquid Crystal Displays (LCD), micro-electro-mechanical-systems (MEMS) based microprojectors and quantum dot displays or combinations thereof may be used to generate the image and/or provide illumination. In certain embodiments, separate means, which may be the same or different, are used for each eye. In other embodiments, a single means is used in combination with additional components for display for both eyes. These additional components may include but are not limited to prisms, mirrors and beam splitters. In certain embodiments, illumination is provided, either fully or in part, by an external light source.

The image capture component is configured to capture images of both eyes, optionally simultaneously. In certain embodiments, the image capture component may include one or more digital cameras. In certain embodiments, a single centrally positioned camera is used. The camera optionally takes both still images and video. In certain embodiments, the image capture component is a high resolution device. In certain embodiments, the capture component detects visible light. In certain embodiments, the capture component detects near infrared light. In other embodiments, the capture component detects both visible and near infrared light.

The device further comprises projection optics which provide magnification/minification of the generated image for the eye; focuses the image at a selected distance; and/or limit the field of view (area of projection) so it is limited to the targeted eye. Accordingly, in certain embodiments the projection optics comprise a focussing element which may contain multiple optical components.

In certain embodiments, the focussing element has a variable focal distance for the full range of refractive error or a selective range of refractive error for human eyes. A focussing element having a variable focal distance allows for the stimulation or control of accommodation and thereby improve measurements of ocular optical properties. In addition, it allows for best corrected visual acuity to be measured. Accordingly, in certain embodiments, the device is a photorefractive aberrometer.

In certain embodiments, the projection optics comprise a lens positioned anterior to the optic components which projects the image from the imaging components at infinity (the lens' power provides an initial magnification and creates an exit pupil for the imaging optics). In this embodiment a telescope comprising two or more telescope lens is positioned anterior to the lens (i.e. between the eye and the lens). The magnification of the telescope is chosen to produce the final targeted magnification when combined with the lens. The position of the telescope is selected so the exit pupil in 1 is imaged in the pupil plane of the eye. The focus can be changed by changing the relative position of the telescope lenses. In this embodiment, an additional lens may be added specifically to control focus. In certain embodiments, translating the additional lens changes the power of the telescope and thus focus point. The additional lens could alternatively be an adaptive lens (such as a liquid lens) which changes shape to change power, it would be positioned at a point which when combined with the other lenses it acts as a telescope when in an average power point, then you can change its power to change the focus point.

In certain embodiments, there are separate projection optics for each eye (OD refers to right eye and OS refers to left eye). By having separate projection optics for each eye, the display is presented to and only to the targeted eye, so input can be controlled for each eye individually. Accordingly, in certain embodiments, the device allows for simultaneous independent images projected to each eye.

In certain embodiments, the device comprises an antenna to measure the electrical signal from the retina to perform electroretinography.

In certain embodiments, the device has wireless communication capabilities. In specific embodiments, the device has cloud access. The cloud may provide secure storage, access to databases, including for example diagnostic databases. In certain embodiments, the device is portable. In specific embodiments, the device is a handheld device.

In certain embodiments, the device comprises an interface, including but not limited to a graphical user interface, that is configured to display device/testing options and/or displays images, such as images of the eyes or face, and/or test results.

In certain embodiments, the device comprises a processor to analyze the data generated.

In certain embodiments, the device of the present invention may be used to perform one or more eye examination tests. Eye examination tests include but is not limited to visual acuity, refraction including objective refraction and subjective refraction, pupil function, ocular motility, visual field, external examination, slit-lamp and retinal examination.

In certain embodiments, the device of the present invention can be used to determine pupil diameter, inter pupil distance, ocular alignment, and refraction. In specific embodiments, the device of the present invention may be used to determine pupil diameter static, inter pupil distance, static ocular alignment, and binocular refraction.

In certain embodiments, the device of the present invention can be used to determine pupil diameter, inter pupil distance, ocular alignment, refraction, accommodation control, aberrations, cover test and visual acuity. In specific embodiments, the device of the present invention can be used to determine pupil diameter static, pupil diameter dynamic, inter pupil distance, static ocular alignment, binocular refraction, accommodation control, binocular aberrations, cover test, best correct visual acuity and uncorrected visual acuity.

In certain embodiments, the device of the present invention can be used to determine pupil diameter, inter pupil distance, ocular alignment, refraction, accommodation control, aberrations, cover test, visual acuity, gross ocular anatomy, high resolution pupil, lens opacities and corneal slit. In specific embodiments, the device of the present invention can be used to determine pupil diameter static, pupil diameter dynamic, inter pupil distance, static ocular alignment, binocular refraction, accommodation control, binocular aberrations, cover test, best correct visual acuity, uncorrected visual acuity, gross ocular anatomy, high resolution pupil, lens opacities and binocular corneal slit.

In certain embodiments, the device of the present invention can be used to determine pupil diameter, inter pupil distance, ocular alignment, refraction, accommodation control, aberrations, cover test, visual acuity, gross ocular anatomy, high resolution pupil, lens opacities, corneal slit and ophthalmoscopy. In specific embodiments, the device of the present invention can be used to determine pupil diameter static, pupil diameter dynamic, inter pupil distance, static ocular alignment, binocular refraction, accommodation control, binocular aberrations, cover test, best correct visual acuity, uncorrected visual acuity, gross ocular anatomy, high resolution pupil, lens opacities, binocular corneal slit and ophthalmoscopy.

In certain embodiments, the device can be used for electroretinography without or with minimal body contact.

In certain embodiments, the device can be used to produce high resolution images for gross and fine anatomy visualization and screening. In certain embodiments, the device can be used to measure corneal thickness centrally or create a full thickness map of the cornea.

In certain embodiments, the device can be used to determine contrast sensitivity, colour testing, determine depth perception and/or dynamic vs static acuity.

In certain embodiments, the device is used for a slit lamp exam. In particular, the bi-ocular projectors project slits on the cornea. By moving the slit image across the means to generate an image, the slit image moves across the cornea, obtaining the same effect as moving the slit mechanically in current devices.

In certain embodiments, the device can be used for impairment screening and/or concussion diagnosis. In certain embodiments, the same device may be used for impairment and concussion assessment. In other embodiments, a device the photorefractive illuminators or the accommodation control is utilized for impairment screening and/or concussion diagnosis.

In these embodiments the device can be used to stress test the visual system by creating visual tasks (pursuit, sorting, identification) that engage higher brain functions and challenge them. The ability to perform these tasks can be monitored through eye reactions (For example, how long before the eye looks at the right target? How much lag between the target position and eye direction during pursuit? What is the eye scanning pattern while examining the image?). In certain embodiments, only one eye is stimulated and the autonomous response in the opposite eye (unstimulated) is examined. For example, light is flashed in one pupil, how is the other pupil responding (i.e. is the other pupil responding as per normal).

The invention is further described with reference to the figures.

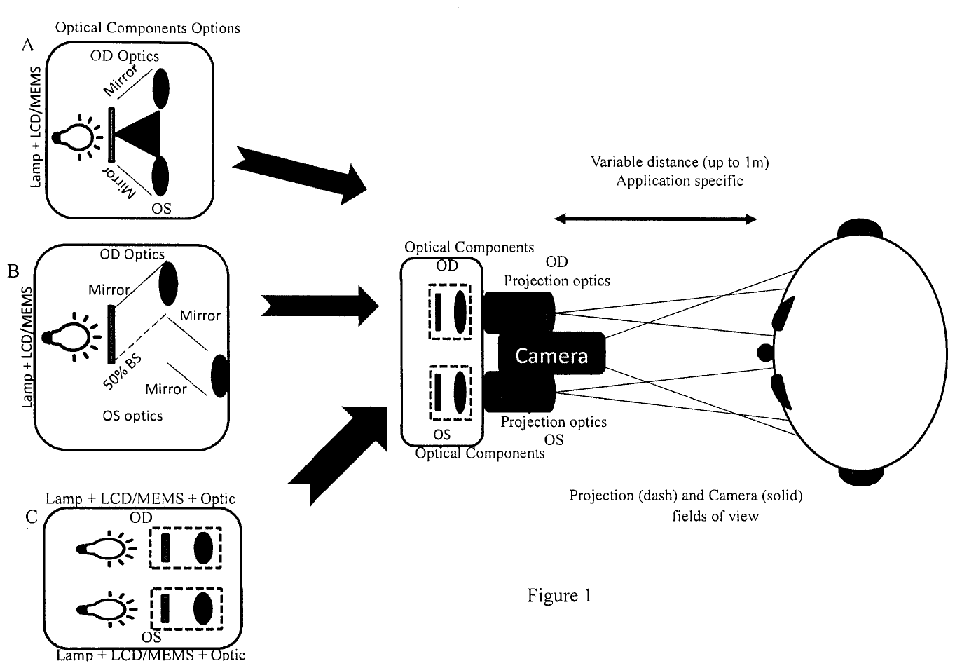

FIG. 1 provides an illustration of the device for visual function testing of various embodiments of the present invention. In particular, the panels labelled A, B and C illustrate various embodiments for the Optical Components of the device of the present invention. In the embodiment illustrated in panel A, a large display and a prism (mirrored surfaces) divides it into a right and left display, and projection optics send the images to each eye. In the embodiment illustrated in panel B, the same image is presented to both eyes, the beam splitter sends 50% to the right eye and the rest to the left eye, and projection optics sends the information to eyes as before. In the embodiment illustrated in panel C—two completely independent Liquid Crystal Displays are utilized.

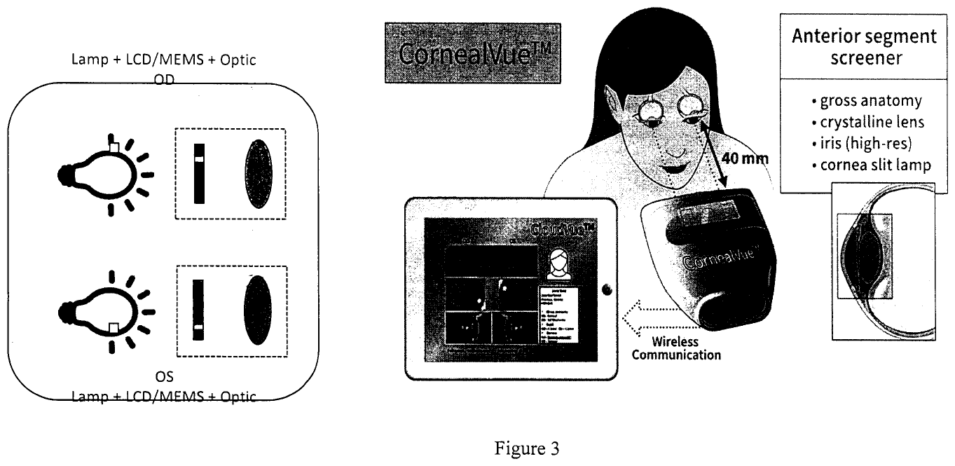

FIG. 3 illustrates device of an embodiment of the present invention which is for use as a slit lamp. In this embodiment, the optical components are setup so that the image is displayed in the cornea/iris region. The examination of the eyes utilizing this device may include gross anatomy examination, detailed examination of the iris and pupils and slit lamp examination.

Gross Anatomy Examination: A high resolution image of the face that include both eyes may be taken in normal lighting. Normal lighting may be provided by using displaying white light from the means to generate an image and/or to provide illumination. The high resolution picture may be used for a gross anatomy examination of the eyes.

Detailed Examination of the Irises and Pupils: A magnified image of the irises and pupil is taken. Magnification may be digital magnification and/or optical. In embodiments in which magnification is optical, the device comprises a magnifying element.

Slit Lamp Examination: Then the displays are made to display a thin white light (adjustable width), the slit, on their targeted eye simultaneously illuminating a specific narrow region of the eye. This provides a cross-sectional view of the cornea and lens in that area. By moving the white line across the display, the eye is scanned and cross-sectional information on the full thickness of the cornea and dome information about the crystalline lens is obtained. The slit images may be collected by video or could be a series of images played as a montage.

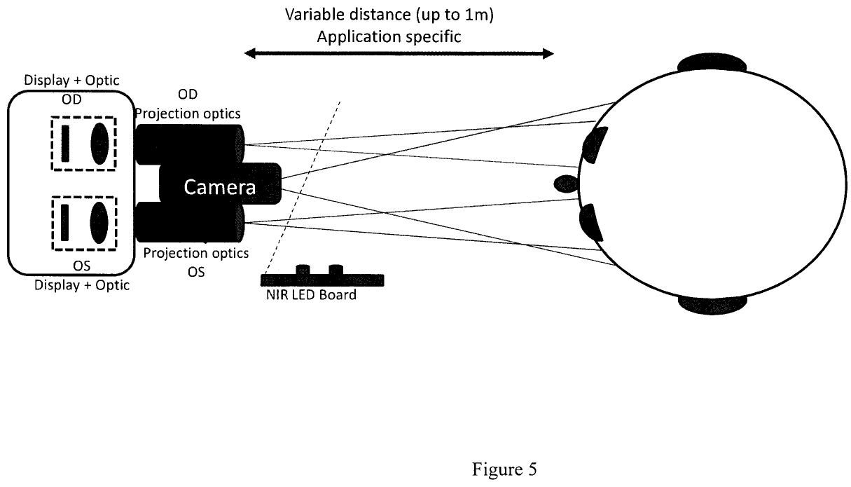

FIG. 5 provides details with respect to the vision screening of an embodiment of the present invention which utilizes NIR LED. The NIR LED illuminates the eyes. Some light enters the eye, is focused on the back of the eye and is scattered back out of the eye. The camera optics are focused on the pupil (as per the vision screener), the light reflected back out appears as a pattern filling partially or completely the image of the pupil (the reflex, often called the photorefractive crescent). The reflex has complex geometry which depends on the optical properties of the eye. The algorithm extracts this optical information from the reflex, part of this information is the prescription but information about aberrations (spherical aberration and coma in particular) is also obtained.

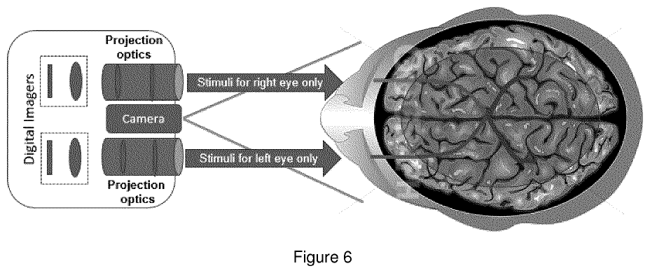

FIG. 6 illustrate application impairment specific testing protocols. For example, the device may illuminate the eyes with white light to test for pupil reaction or test for an offset between right and left eyes to test for stereovision. Impairment may be tested by challenging the brain with complex tasks or stimuli.

What is claimed is:

1. A device for visual function testing comprising optic components, projection optics and an image capture component.

2. The device of claim 1, wherein the optical components comprise one or more means to generate an image and/or to provide illumination.

3. The device of claim 2, wherein the one or means are selected from the group consisting of LED displays, Organic LED (OLED) displays, Liquid Crystal Displays (LCD), micro-electro-mechanical-systems (MEMS) based microprojectors, quantum dot displays and combinations thereof may be used to generate the image and/or provide illumination.

4. The device of claim 2, wherein separate means are used to generate the image and/or provide illumination are used for each eye.

5. The device of claim 2, wherein a single means is used in combination with additional components for display for both eyes, and wherein said additional components comprise prism(s), mirror(s) and/or beam splitter(s).

6. The device of claim 1, wherein said image capture component is configured to capture images of both eyes simultaneously.

7. The device of claim 1, wherein said image capture component comprises one or more digital cameras.

8. The device of claim 1, further comprising projection optics which provide magnification/minification of the generated image for the eye; focuses the image at a selected distance; and/or limit the field of view (area of projection) so it is limited to the targeted eye.

9. The device of claim 8, wherein the projection optics comprise a focussing element.
